(19) United States
(12) Patent Application Publication
(10) Pub. No.: US 2025/0339394 A1
(43) Pub. Date: Nov. 6, 2025

(54) COMPOSITIONS COMPRISING ENCAPSULATED TRETINOIN

(71) Applicant: Mayne Pharma LLC, Raleigh, NC (US)

(72) Inventors: Ofer TOLEDANO, Kfar Saba (IL); Karine Neimann, Ness Ziona (IL); Maya Erlich, Ness Ziona (IL); Dorit Marco, Rehovot (IL)

(73) Assignee: Mayne Pharma LLC, Raleigh, NC (US)

(21) Appl. No.: 19/267,732

(22) Filed: Jul. 14, 2025

Related U.S. Application Data
(63) Continuation of application No. 16/033,260, filed on Jul. 12, 2018, now Pat. No. 12,357,602.
(60) Provisional application No. 62/531,402, filed on Jul. 12, 2017.

(51) Int. Cl. A61K 31/202; A61K 9/00; A61K 9/06; A61K 9/107; A61K 9/50; A61K 31/203

(52) U.S. Cl. CPC A61K 31/202; A61K 9/0014; A61K 9/06; A61K 9/107; A61K 9/501; A61K 31/203

(57) ABSTRACT

The present application is directed to compositions comprising microcapsules comprising encapsulated tretinoin, wherein the microcapsule size is less than 50 μm; and to methods of use thereof.

### CROSS REFERENCE TO RELATED APPLICATIONS

This application is a Continuation Application of U.S. application Ser. No. 16/033,260, filed on Jul. 12, 2018, which claims the benefit of U.S. Ser. No. 62/531,402, filed on Jul. 12, 2017, which are incorporated in their entirety herein by reference.

### FIELD OF THE INVENTION

The present application is directed to compositions comprising encapsulated tretinoin.

### BACKGROUND OF THE INVENTION

Topical retinoids are keratinization inhibitors. They work by decreasing the cohesiveness of follicular epithelial cells. This, results in an inhibition in the formation of microcomedones, preventing the formation of mature comedones and inflammatory lesions (Gollnick and Cunliffe, J. Am. Acad. Dermatol 2003; 49: S1-38). Use of retinoids promotes the normal desquamation of follicular epithelium. The action of the retinoid may enhance the penetration of other topical compounds used to treat acne.

BPO is a commonly used topical antibacterial agent for acne available either by prescription in combinations or over the counter (OTC). BPO has been found to be lethal to P. acnes as well as other bacteria that may reside on the skin. So far there has been no indication of any bacteria developing a resistance to BPO. It has also been demonstrated that BPO has keratolytic activity contributing to its efficacy in treating comedonal acne (Tanghetti, Cutis, 2008, 82(5S), 3-11). BPO reduces the cohesiveness of the cells of the stratum corneum, thus improving topical drug delivery through the epidermal barrier.

Silica microcapsule systems have been developed to overcome many of the limitations (such as degradation and irritation) of standard pharmaceutical formulations involving multiple active ingredients. The encapsulation of active ingredients in silica microcapsules serves to protect components in the formulation from interacting with one another and, as a consequence, increases overall formulation stability. Silica is chemically inert, photochemically and physically stable, and safe for topical use.

Clinicians have been reluctant to prescribe topical retinoids and BPO concurrently due to a belief that the BPO may result in oxidation and degradation of the tretinoin molecule, thereby reducing its effectiveness, and prefer to recommend the BPO or an antibiotic/BPO combination to be applied in the morning and tretinoin at night (Yan A C. Adolesc. Med. Clin. 2006; 17(3):613-637).

Another publication (Emmy Graber, Treatment of Acne Vulgaris, UpToDate.com, July 2016) states “topical tretinoin should NOT be applied at the same time as benzoyl peroxide”, despite the known fact that newer retinoid compositions like Retin A microspheres (MICROSPONGE® System) have less interaction or no short-term interaction with BPO. Obviously, concomitant administration of tretinoin and BPO is taught away by this publication.

BPO is known to oxidize tretinoin and hence it was feared that their interaction on the skin when administered together will diminish the therapeutic effect of tretinoin. Thus, while there are some reports in the literature on the value of both compounds being administered one in the morning and the other in the evening, the verdict up to now was that the two products should not be administered concomitantly.

This belief of the medical profession explains why all previous attempts to solve the stability problem of tretinoin/BPO, such as microencapsulation technology, did not yield a commercial product so far.

Since topical conditions such as acne has multiple pathogenic factors, such as abnormal follicular keratinization, P. acnes proliferation and inflammation, combining separate active agents that target these multiple factors would provide the patient with an effective and convenient treatment improving treatment outcomes.

### SUMMARY OF THE INVENTION

The inventors of the present invention have found that in order for topical treatment with tretinoin to be effective, especially in combination with BPO, the dissolution rate of the tretinoin component should be reduced to less than 60% wt/h. This is achievable by designing the microcapsules encapsulating solid tretinoin to have a size of less than 50 μm, according to the present invention.

In some embodiments, the present application is directed to an encapsulated tretinoin composition, said composition comprising microcapsules comprising a core comprising tretinoin coated by a shell, wherein said core is in the solid form and said microcapsules have a size of less than about 50 μm.

In other embodiments, the present invention is directed to an encapsulated tretinoin composition, said composition comprising microcapsules comprising a core comprising tretinoin coated by a shell, wherein said core is in the solid form and said microcapsules have a size of less than about 50 μm; and wherein the concentration of all-trans 5,6-epoxy retinoic acid is lower than 1% after two weeks storage at 40° C., in the presence of benzoyl peroxide.

In some embodiments, the present application is directed to an encapsulated tretinoin composition, said composition comprising microcapsules comprising a core comprising tretinoin coated by a shell, wherein said core is in the solid form and said microcapsules have a size of less than about 50 μm, and said composition further comprises encapsulated or non-encapsulated benzoyl peroxide. In another embodiment, the benzoyl peroxide is encapsulated.

In some embodiment, this invention provides an encapsulated tretinoin composition, said composition comprising microcapsules comprising a core comprising tretinoin coated by a shell, wherein said core is in the solid form and said microcapsules have a size of less than about 50 μm; and said composition has tretinoin dissolution rate of less than about 60% weight/h as measured in a medium of 30%:70% V/V mixture of water and isopropyl alcohol at ambient temperature. In other embodiments, the tretinoin dissolution rate is of less than about 40% weight/h.

In other embodiments the composition of this invention comprises microcapsule comprising an encapsulated tretinoin, wherein the microparticle size is between about 5 μm to about 45 μm. In another embodiment between about 30 μm to about 50 μm.

In some embodiments, the compositions of this invention comprise all-trans 5,6-epoxy retinoic acid at a concentration of less than 1%. In other embodiments, at a concentration of less than 0.7%. In some embodiments the degradation of the tretinoin from the composition of this invention is less than 2.5% after two weeks storage at 40° C. In another embodiment, the degradation of said tretinoin is less than 2%.

In some embodiments, the concentration of tretinoin in the composition of this invention is between about 0.01% to about 0.1% weight of the composition. In some embodiments, the concentration of tretinoin in the composition of this invention is between about 0.05% to about 0.1% weight of the composition. In some embodiments, the concentration of tretinoin in the composition of this invention is about 0.075% weight of the composition. In some embodiments, the concentration of benzoyl peroxide in the composition of this invention is about 3% weight of the composition.

In some embodiments, this invention provides a method for treating a surface condition in a subject in need thereof, said method comprising topically administering to said subject a composition of this invention. In other embodiments, the surface is skin or mucosal membrane. In other embodiments, the surface condition is a skin disease, disorder or condition selected from acne, infection, inflammation, pruritus, psoriasis, seborrhea, contact dermatitis, rosacea, and a combination thereof.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which

FIG. 1 shows the HPLC chromatogram of an embodiment composition of the invention comprising 0.05% E-ATRA and 3% E-BPO eluted with acetonitrile and acetic acid 1% in water on a Zorbax RX-C18 3.5 mμ, 4.6*75 mm column, showing the RRT 0.44 product (all-trans 5,6-epoxy retinoic acid at retention time of about 3.5 min (RRT product calculated relative to the ATRA peak at 7.8 min).

### DETAILED DESCRIPTION OF THE PRESENT INVENTION

Thus, in the first aspect, the invention provides a composition comprising tretinoin encapsulated in the core of a microcapsule having a shell, wherein said core is in the solid form and wherein said microcapsule size is less than about 50 μm. In another aspect this invention provides an encapsulated tretinoin composition, said composition comprising microcapsules comprising a core comprising tretinoin coated by a shell, wherein said core is in the solid form and said microcapsules have a size of less than about 50 μm.

As used herein unless otherwise indicated the term “microcapsule” refers to a microparticle having a core shell structure, wherein said core comprises an active agent as defined herein (tretinoin), being coated by a shell forming the microcapsule entrapping the core.

In some embodiments, tretinoin is defined as the core material, i.e. the core of said microcapsule consists of tretinoin alone. In other embodiments, said core of said microcapsule comprises an active agent and at least one Phase Changing Material (PCM), i.e. under these embodiments the core material comprises both the active agent and the PCM. In some embodiments, the coating/shell is directly deposited on the core material. In some embodiments, the coating/shell is directly deposited on the tretinoin forming the core material of said microcapsule. In some embodiments, the core material is solid. In other embodiments, the core material is semi-solid. In some embodiments, the core material consists of a solid particle of the active agent. In other embodiments, the core material comprises a solid particle of the active agent.

In the context of the present invention, the term “core” and/or “core material” used interchangeably herein, refers to the inside/internal part of the microcapsules comprising said active agent, and, in some embodiments also said at least one phase changing material. The core or core material is surrounded by said shell of said microcapsule. It should be noted that additional compounds may be present in said core including for example carriers, excipients, pharmaceutically acceptable polymers or salts etc., all in accordance with the intended use of produced microcapsules, which will be apparent to a skilled artisan preparing said microcapsules.

In some embodiments, the present invention a process for obtaining a thick and dense coating on said core/core material, using in some embodiments metal oxide nanoparticles in combination with a sol-gel precursor, wherein the addition of phase changing material incorporated into said core provides further stability parameters to the encapsulated active agents and to the pharmaceutical composition comprising them.

Thus, in some embodiments of the present invention, there is provided a process for preparing microcapsules having a core encapsulated within a metal oxide shell, said process comprising: (a) preparing an oil-in-water emulsion by emulsification of an oily phase comprising at least one active agent and at least one phase changing material, in an aqueous phase, wherein at least one of said oily phase and aqueous phase comprise a sol-gel precursor; (b) subjecting said emulsion to microcapsule forming conditions; thereby obtaining said microcapsules.

In other embodiments of the invention said core may be solid at room temperature. In other embodiments, said core may be in a semi-solid phase at room temperature. The oily phase utilized by a process of the invention comprises at least one active agent and at least one phase changing material. Said at least one active agent may be in a form of a water insoluble liquid or dispersion in water-insoluble liquid comprising said at least one active agent. The oily phase may be constituted by a liquid water-insoluble active agent; which may comprise a first, liquid water-insoluble active agent dissolved and/or dispersed in a second, water insoluble liquid being another active agent or serving as a carrier. In another embodiment said oily phase may comprise a solid active agent dissolved and/or dispersed in a water-insoluble liquid, being another active ingredient or serving as a carrier. The term “water insoluble liquid” or “dispersion in water-insoluble liquid” refers to a solubility of the liquid (including the ingredients included therein, dissolved and/or dispersed) in water of about less than 1% w/w, preferably 0.5% w/w and most preferably 0.15% w/w at room temperature (20-25° C.). Accordingly, the constituents included in the core whether solid or liquid ingredients have a solubility of about less than 1% w/w, preferably 0.5% w/w and most preferably 0.15% w/w at room temperature (20-25° C.). The water insoluble liquid may be for example squalane oil, polydimethylsiloxane, mineral oil, castor oil, aromatic 200, and mixtures thereof.

In some embodiments the viscosity of said core/core material of said microcapsule (at room temperature) may be about 300 cP, 350 cP, 400 cP, 450 cP, 500 cP, 550 cP, 600 cP, 650 cP, 700 cP, 750 cP, 800 cP, 900 cP, 1000 cP, 2000 cP, 3000 cP, 4000 cP, 5000 cP, 6000 cP, 7000 cP, 8000 cP, 9000 cP, 10,000 cP, 20,000 cP, 30,000 cP, 40,000 cP, 50,000 cP, 60,000 cP, 70,000 cP, 80,000 cP, 90,000 cP, 100,000 cP, 200,000 cP, 300,000 cP, 400,000 cP, 500,000 cP, 600,000 cP, 700,000 cP, 800,000 cP, 900,000 cP or I,000,000 cP (when measured under various conditions). In some embodiments, the viscosity of said core at room temperature is between about 300 to 600 cP. In other embodiments, the viscosity of said core at room temperature is between about 400 to 500 cP. In further embodiments, the viscosity of said core at room temperature is between about 300 to 10,000 cP. In other embodiments the viscosity of said core at room temperature is between about 5,000 to I,000,000 cP. In some further embodiments the viscosity of said core at room temperature is between about 20,000 to I,000,000 cP.

Further input regarding the process of obtaining a core stabilized microcapsule can be found in the International publication WO 2011/080741, which is herein incorporated by reference in its entirety.

In one embodiment, said at least one phase changing material is selected from natural or synthetic paraffins (e.g. having a molecular formula of CₙH₂ₙ₊₂, wherein n=10-100), C₁₀-C₁₀₀ alkane, C₁₀-C₁₀₀ alkene (having at least one double bond), C₁₀-C₁₀₀ alkyne (having at least one triple bond), aliphatic alcohols (e.g. having a molecular formula of CH₃(CH₂)ₙOH, wherein n=10-100) and fatty acids (e.g. having a molecular formula of CH₃(CH₂)₂ₙCOOH, wherein n-10-100), or any combination thereof.

In some embodiments, said at least one phase changing material is at least one natural or synthetic paraffin. In some embodiments, said at least one phase changing material is a C₁₀-C₁₀₀ aliphatic alcohol (in other embodiments C₁₀, C₂₀, C₃₀, C₄₀, C₅₀, C₆₀, C₇₀, C₈₀, C₉₀ to C₁₀₀ aliphatic alcohol). In further embodiments, said at least one phase changing material is a C₁₀-C₁₀₀ aliphatic fatty acid (in other embodiments C₁₀, C₂₀, C₃₀, C₄₀, C₅₀, C₆₀, C₇₀, C₈₀, C₉₀ to C₁₀₀ aliphatic fatty acid).

In one embodiment said PCMs are liquified (or at least become substantially or partially liquified, pliable or semi-solid, and capable of being handled by a process of the invention) at a temperature range of between about 35° C. to about 60° C., more preferably in a temperature range of between about 35° C. to about 45° C.

Examples of phase changing materials capable of being used by the processes of the invention include, but are not limited to: Carnauba wax (m.p. 82-86° C.), Beeswax pure (m.p. 61-65° C.), Beeswax white pure, (m.p. 61-65° C.), Beeswax bleached technical (m.p. 61-65° C.), Montan wax bleached (m.p. 80-86° C.), Montan wax bleached, partially saponified (m.p. 99-105° C.), Montanic acid (m.p. 81-87° C.), Hydrocarbon wax synthetic (m.p. 106-114° C.), Microcrystalline wax (m.p. 89-95° C.), Microcrystalline wax (m.p. 76-82° C.), Hardwax partially saponified (m.p. 104-109° C.), Beeswax yellow (m.p. 61-66° C.), Polishing Wax (m.p. 78-84° C.), Castor wax (m.p. 83-89° C.), Microwax (m.p. 89-95° C.), Microwax (m.p. 80-86° C.), Microwax (m.p. 76-82° C.), Ozokerite (m.p. 72-79° C.), Microcrystalline wax, plastic (m.p. 76-82° C.), Microcrystalline wax, soft (m.p. 74-80° C.), Wax blend (m.p. 62-68° C.), Polyolefin wax (m.p. 65-75° C.), Lanolin, Shellac, Bayberry wax (m.p. 45° C.), Candelilla wax (m.p. 67-79° C.), Ouricury wax, Rice bran wax (m.p. 77-86° C.), Soy candle (wax), Paraffin (m.p. 47-64° C.), Chinese wax, and any combinations thereof.

In one embodiment of a process for the preparation of a microcapsule, said at least one phase changing material is in a liquid state. Thus, prior to the addition of said at least one PCM, its temperature is raised until it is substantially homogenously liquified. In a further embodiment of the present invention, a process of the invention is carried out under a temperature wherein said at least one phase changing material is in a liquid state, throughout the entire emulsification and encapsulation process disclosed herein above and below. It is noted that said at least one PCM utilized by a process of the present invention, is selected such that its heat of fusion allows for processes of the invention to be carried out substantially without compromising the active agents used, the emulsion formed and the metal oxide shell produced for the microcapsules of the invention.

In one embodiment of a process for the preparation of a microcapsule, at least one metal oxide nanoparticle is added to said aqueous phase prior, during or after emulsification of step (a).

In a further embodiment of a process for the preparation of a microcapsule, the process further comprises a step of cooling obtained microcapsules to room temperature. It is noted that upon cooling of said obtained microcapsules, the viscosity of said core, comprising said at least one active agent and at least one PCM, changes to have values of between about 300 cP to I,000,000 cP (when measured under various conditions). It should be understood that such PCMs used by a process of the invention are accumulated in the core of obtained microcapsules and are not incorporated in any part of the metal-oxide shell formed by encapsulation process of the invention.

The size of the microcapsules (denoted herein also by the general term “particles” or “microparticles”) as will be referred to herein refers to D₉₀ meaning that 90% of the particles have the stated dimension or less (measured by volume). Thus, for examples, for spherical particles stated to have a diameter of less than about 50 μm (“microns”), this means that the particles have a D₉₀ of 50 microns. The D₉₀ (termed also d(0.9)) may be measured by laser diffraction. For particles having a shape other than spheres, the D₉₀ refers to the mean average of the diameter of a plurality of particles.

In some embodiments, said microcapsule size is less than about 45 μm. In other embodiments, said microcapsule size is between about 5 μm to about 45 μm. In other embodiments, said microcapsule size is between about 30 μm to about 50 μm. In other embodiments, said microcapsule size is between about 30 μm to about 45 μm. In certain embodiments, the microcapsule size is about 35 μm to about 45 μm.

A composition according to the present invention, wherein the shell of the microcapsules is an inorganic polymeric shell. In some embodiments, the shell is a metal oxide or semi-metal oxide shell. In some embodiments, the metal oxide or semi-metal oxide shell is formed by a sol-gel encapsulation/coating process.

In some embodiments, the metal oxide is selected from silica, titania, alumina, zirconia, ZnO, and mixtures thereof. In some other embodiments, the metal oxide is silica.

According to certain embodiments of the present invention, the surface of the metal oxide layer of the coated particulate matter may be chemically modified by organic groups, in some embodiments hydrophobic groups, attached to its surface. The hydrophobic groups may be for example alkyl groups (such alkyl groups may be further substituted with one or more fluoro atoms), aryl groups (such as benzyl or phenyl), and combinations thereof. The groups may be as described below with respect to the process.

In some embodiments, the microcapsules are formed using a process as disclosed in the following documents (herein incorporated by reference): U.S. Pat. Nos. 6,303,149, 6,238,650, 6,468,509, 6,436,375, US2005037087, US2002064541, and International publication Nos. WO 00/09652, WO00/72806, WO 01/80823, WO 03/03497, WO 03/039510, WO00/71084, WO05/009604, and WO04/81222, disclose sol-gel microcapsules and methods for their preparation; EP 0 934 773 and U.S. Pat. No. 6,337,089 teach microcapsules containing core material and a capsule wall made of organopolysiloxane, and their production; EP0941 761 and U.S. Pat. No. 6,251,313 also teach the preparation of microcapsules having shell walls of organopolysiloxane; U.S. Pat. No. 4,931,362 describes a method of forming microcapsules or micromatrix bodies having an interior water-immiscible liquid phase containing an active, water-immiscible ingredient. Microcapsules prepared by a sol-gel process are also disclosed in GB2416524, U.S. Pat. No. 6,855,335, WO03/066209.

According to some embodiments of the present invention, the coated form of the active ingredient (microcapsule) may be in form of a polymeric microsponge/silica microsphere where the active ingredient is adsorbed, embedded, impregnated or entrapped in the microsponge/silica microshpere as described for example in U.S. Pat. Nos. 4,690,825; 5,145,675, 5,879,716, 5,955,109, and 9,452,137 incorporated herein by reference in their entirety.

In other embodiments, microcapsules are formed by the encapsulation process disclosed in the following publications (herein incorporated by reference): U.S. Pat. Nos. 7,629,394, 9,205,395, US 2015/0328615, US 2014/0186630. Controlled release microcapsules: IN01958CH2007, IN02080CH2007, U.S. Pat. Nos. 4,235,872, 4,670,250, EP 0248531, U.S. Pat. Nos. 4,970,031, 5,238,714, WO9321764, U.S. Pat. No. 5,575,987, WO9420075, US 2004/137031, US 2006/003014, US 2010/180464.

Further according to an embodiment of the present invention the obtained metal oxide coating layer has a width (thickness) of 0.1 μm or above, in some embodiments the metal oxide coating layer has a width (thickness) of 0.1-10 μm.

Additionally, according to an embodiment of the present invention the obtained metal oxide coating layer has a width (thickness) of 0.3 μm or above, in some embodiments the metal oxide coating layer has a width of 0.3-10 μm.

Additionally, according to an embodiment of the present invention, the thickness of the metal oxide layer is in the range of 0.1-10 μm. In some further embodiments, the thickness of the metal oxide layer is in the range of 0.1-3 μm, and in some further embodiments in the range of 0.1-1 μm. The thickness of the metal oxide layer may also be in some embodiments in the range of 0.3 to 3 μm, and in some other embodiments in the range of 0.3 to 2 μm.

Further according to an embodiment of the present invention the obtained metal oxide coating layer has a width (thickness) of about 0.1, 0.2, 0.3, 0.5, 0.7, 1, 1.5, 2 or 5 μm or above, in some embodiments up to 10 μm.

The width of the metal oxide layer may be determined for example by a Transmission Electron Microscope or Confocal Microscope such that in a circular cross sectional area of the particle the smallest width is at least e.g. 0.1 μm (the width is determined as the smallest distance from the surface of the particle (i.e. metal oxide surface) to the core-metal oxide interface).

The microcapsules are in some embodiments characterized in that the core material is substantially free of the metal oxide and further in that the metal oxide layer is substantially free of the core material, e.g. either as particle dispersion (in the nano-metric range of below 0.1 μm) of the particulate matter or as molecular dispersion of the particulate matter.

Thus, according to an embodiment of the present invention, the metal oxide layer is substantially free of core material (either in the form of molecules or as nano-metric particles). The term “substantially free” in this context denotes that the concentration of the molecules of the core material or the concentration of the nano-metric particles of the core material is negligible as compared to the metal oxide. Similarly, by the term “the core material is substantially free of the metal oxide” is meant that the concentration of the metal oxide in the core is negligible as compared to the core material.

According to another embodiment when the microcapsules are prepared by a method such as spray drying, the core material comprising the active agent may further comprise up to about 30% w/w, in some embodiments up to about 20% metal oxide and the metal oxide coating layer may further comprise up to about 30% w/w, in some embodiments up to about 20% w/w of the active agent.

According to an embodiment of the present invention the weight ratio of the metal oxide to the solid particulate matter is in the range of 1:99 to 50:50. The weight ratio of the metal oxide layer to the solid particulate matter may be also in the range of 3:97 to 50:50, 5:95 to 50:50, 10:90 to 50:50, 5:95 to 30:70, 10:90 to 30:70. Further, according to an embodiment of the present invention the rate ratio of the metal oxide to the solid particulate matter is in the range of 10:90 to 20:80.

According to another embodiment of the present invention, when spray drying method is used, the weight ratio of the metal oxide to the solid particulate matter may be in the range 5:95 to 95:5.

In some embodiments, said composition of the invention has tretinoin dissolution rate of less than about 60% weight/h as measured in a medium of 30%:70% V/V mixture of water and isopropyl alcohol at ambient temperature. In some further embodiments, said tretinoin dissolution rate is less than about 40% weight/h.

It should be noted that the dissolution rate (release rate) defined herein relates to the measurement (either in vitro or in vivo) of the rate at which the active agents (tretinoin) is released from the topical medicament of the invention, to the extracting media or skin. The release rate is measured using known method as defined herein, i.e. 70% IPA (isopropyl alcohol) and 30% water and optionally an antioxidant (such as BHT) at room temperature.

In some embodiments, a composition of the invention further comprises benzoyl peroxide (BPO), as a second active agent.

In some embodiments, said BPO is non-encapsulated in a composition of the invention. In another embodiments, said BPO is encapsulated in a separate microcapsule (i.e. “BPO microcapsule”, a microcapsule that is different and separated from the microcapsule encapsulating the tretinoin (“tretinoin microcapsule”) in a composition of the invention). In some embodiments, said microcapsule encapsulating said BPO has a shell different than the shell of said tretinoin microcapsule. In other embodiments, said microcapsule encapsulating said BPO has a shell similar to the shell of said tretinoin microcapsule (i.e. it is formed using similar encapsulating processes or similar encapsulating reagents). In some embodiments, the shell of said BPO microcapsule is a metal oxide shell. In some embodiments, said BPO is in the solid form. In other embodiments, said BPO microcapsule has a solid core. In other embodiments, said BPO microcapsule consists of solid BPO. In other embodiments, said shell of said BPO microcapsule is directly deposited on solid BPO.

As used herein by the term “non-encapsulated form” or “non-coated form” is meant that the active ingredient (BPO) is present in the composition in its “naked” form meaning that it is not intimately embedded, encapsulated, entrapped or encased in a polymeric carrier, and is present in the composition in direct contact with the composition carrier. As used herein by the term “coated form of the active ingredient” is meant that the active ingredient is embedded, dispersed, entrapped, or encased, e.g. as a solid dispersion or molecular dispersion in a polymeric carrier which may be an organic or inorganic carrier and which may serve as a matrix for dispersing the active ingredient or as encapsulated material coating said active ingredient (i.e the active ingredient is present in a core or is a core material encapsulated by a shell composed of a polymeric material which may be an organic or inorganic polymer).

In some embodiments wherein said composition of the invention further comprises BPO, the concentration of RRT (relative retention time) 0.44 (all-trans 5,6-epoxy retinoic acid) is lower than 1% after two weeks storage at 40° C. In other embodiments, the concentration of RRT 0.44 (all-trans 5,6-epoxy retinoic acid) is lower than 0.7% after two weeks storage at 40° C.

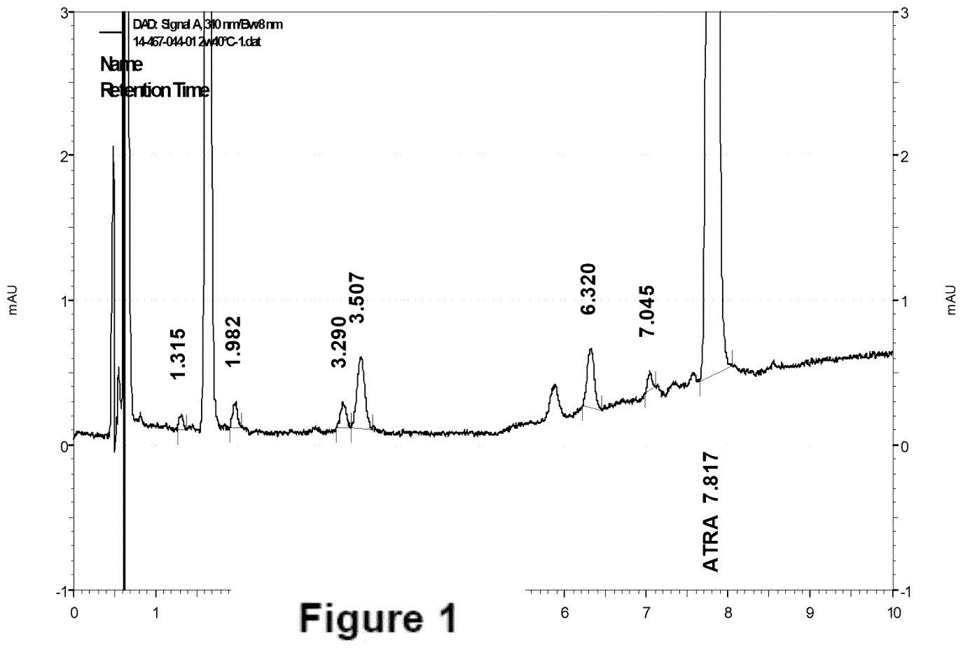

When referring to RRT 0.44 it should be understood to relate to the degradation product of tretinoin in the presence of BPO as shown in the HPLC chromatography of the composition of the invention after two weeks of storage at 40° C. An example of the RRT product can be seen in FIG. 1 at retention time 3.507 min. In other embodiments, RRT 0.44 refers to all-trans 5,6-epoxy retinoic acid (CAS 13100-69-1) represented by the following structure:

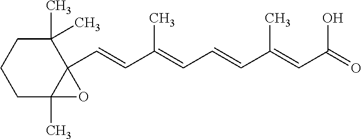 [SMILES: CC(/C=C/C12OC1(C)CCCC2(C)C)=C\C=C\C(C)=C\C(=O)O]

In some embodiments wherein said composition of the invention further comprises BPO, the degradation of said tretinoin is less than 2.5% after two weeks storage at 40° C. In other embodiments, the degradation of said tretinoin is less than 2%.

In some embodiments, wherein the composition of the invention further comprises BPO, the degradation of said tretinoin is not more than 5% after 18 or 24 months storage at 2° C.-8° C. In other embodiments, the degradation of said tretinoin is not more than 5% after 3 months storage at 25° C. and 60% RH.

In some embodiments, said medicament is administered in a single composition, single fixed dose medicament, comprising both said active agents (BPO and tretinoin). The term “fixed dose medicament” should be understood as meaning a combination whose active agents are combined at fixed doses in the same vehicle (single formula) that delivers them together to the point of application.

In further embodiments, said medicament comprises two separate compositions each one comprising each of said active agents. In such embodiments the weight % amount of each active agent relates to each of their weight amount in each composition separately. In some embodiments, said two separate compositions of said medicament are administered concomitantly. In further embodiments, said two separate compositions are administered sequentially.

In some embodiments, a composition of the invention comprises tretinoin is in the amount of between about 0.01% to about 0.1% weight of the composition.

In some embodiments, a composition of the invention comprises tretinoin is in the amount of between about 0.05% to about 0.1% weight of the composition.

In some embodiments, a composition of the invention comprises tretinoin is in the amount of about 0.075% weight of the composition.

In other embodiments, a composition of the invention comprises BPO is an amount of about 3% weight of the composition.

In another embodiment, a composition of the invention comprises tretinoin in an amount of about 0.1% weight and benzoyl peroxide in an amount of about 3% weight. In another embodiment, a composition of the invention comprises tretinoin in an amount of about 0.05% weight and benzoyl peroxide in an amount of about 3% weight.

In one embodiment, the term “about”, refers to a deviance of between 0.0001-5% from the indicated number or range of numbers. In one embodiment, the term “about”, refers to a deviance of between 1-10% from the indicated number or range of numbers.

In some embodiments, the composition of this invention is a pharmaceutical composition. The term “pharmaceutical composition” means a composition suitable for pharmaceutical use as defined herein. In another embodiment, the pharmaceutical composition comprises a suitable carrier or diluent. The pharmaceutical composition of this invention includes a therapeutically effective amount of the active ingredient, i.e. the amount which provides a therapeutic effect for a given condition and administration regimen.

The active component can be formulated into the composition as its hydrate, solvate, or as its pharmaceutically acceptable salt. Suitable pharmaceutically acceptable salts of the active component(s) (i.e. tretinoin) of this invention include inorganic salts such as: ammonium, alkali metals to include lithium, sodium, potassium, cesium; alkaline earth metals to include calcium, magnesium, aluminium; zinc, barium; or quaternary ammoniums; or organic salts such as arginine, organic amines to include aliphatic organic amines, aromatic amines, t-butylamines, (N-benzylphenethylamine), dicyclohexylamines, dimethylamines, diethanolamines, ethanolamines, ethylenediamines, imidazoles, lysines, methylamines, N-methyl-D-glucamines, N,N′-dibenzylethylenediamines, pyridines, picolinates, piperazines, tris(hydroxymethyl) methylamines, triethylamines, triethanolamines, trimethylamines, or ureas.

In some embodiment, a composition of the invention is a topical medicament. The term “topical medicament” as used herein should be understood to encompass any pharmaceutical formulation that enables the administration of the active agents to a skin tissue of a patient administered with said medicament. The composition or topical medicament of the present invention comprises a carrier. According to an embodiment of the present invention the carrier is in the form of an ointment, a cream, a lotion, an oil, a solution (in some embodiments an aqueous solution), an emulsion, a gel, a paste, a milk, an aerosol, a powder, or a foam, each represents another embodiment of this invention. In some embodiments, the carrier is an aqueous-based carrier (such as a gel, oil-in water emulsion or oil-in water cream, aqueous solution, foam, lotion, spray).

In yet another aspect, the present invention provides a method for treating a surface condition (e.g., a skin disease or disorder) in a subject in need thereof, comprising topically administering to the subject an effective amount of a composition as described herein. In certain embodiments, the surface is skin or mucosal membrane. In some embodiments, the surface condition is a skin disease, disorder or condition selected from acne, infection, inflammation, pruritus, psoriasis, seborrhea, contact dermatitis, rosacea, melasma, photoaging, photodamage, fine wrinkles, and a combination thereof, each represents another embodiment of this invention. In another embodiment, the method of this invention is directed for treating acne.

The term “treating” or “treatment” as used herein includes any treatment of a condition, disease or disorder associated with a patient's body surface such as the skin or mucosal membrane, and includes inhibiting the disease or disorder (i.e. arresting its development), relieving the disease or disorder (i.e. causing regression of the disease or disorder), or relieving the conditions caused by the disease (i.e. symptoms of the disease). The concentrations of the dermatological agents that can be used for treatment of a specific disease or disorder may be as described in The Merck index an encyclopedia of chemical drugs and biologicals, Rahway, N.J.; Merck & Co; 1989, incorporated herein by reference in its entirety.

Although individual needs may vary, determination of optimal ranges for effective amounts of the compositions is within the skill of the art. Generally, the dosage required to provide an effective amount of a pharmaceutical composition, which can be adjusted by one skilled in the art, will vary depending on the age, health, physical condition, weight, type and extent of the disease or disorder of the recipient, frequency of treatment, the nature of concurrent therapy (if any) and the nature and scope of the desired effect(s).

Thus, the final form of the composition may be any of the above forms, mentioned with respect to the carrier, where the microcapsules are dispersed in the carrier. The final form of the composition may also be in the form of a wash or cleanser.

The following examples are presented in order to more fully illustrate the preferred embodiments of the invention. They should in no way, however, be construed as limiting the broad scope of the invention.

### EXAMPLES

### Example 1

### Preparation of Formulation of Encapsulated ATRA (0.05%) and Encapsulated BPO (3%)

### Oil Phase

2000.0 grams of Cyclomethicone 5N, 1500.0 grams of Cetyl Alcohol, and 1500.0 grams of Mono- and di-Glycerides were mixed and heated to 70° C. until melting. 200.0 grams of Carbopol 980 NF were added and the oily phase was mixed and heated until addition to the main reactor.

### Water Phase

Weight in 50 L Reactor (main reactor)

50.0 grams of Ethylenediaminetetraacetate Disodium salt, 1000.0 grams of Macrogol Stearate Type I, 2000.0 grams of Glycerin (99.5%) and 125.0 grams of Imidurea were dissolved into 29.0 kilograms of water. The solution was mixed and heated to 65° C. until addition of the oily phase to the main reactor.

### E-BPO/Citric Acid Phase

100.0 grams of Anhydrous Citric Acid were added into 10,000.0 grams of 15% encapsulated benzoyl peroxide water suspension. The suspension was mixed and heated to 45-50° C. until addition to the main reactor.

### Emulsion: Main Reactor

The oily phase was added to the water phase under circulation and mixing at 65° C. for 5 minutes. The resulting emulsion was homogenized, mixed and circulated under vacuum for 20 minutes then additional 10 minutes after adding 200.0 grams of NaOH 20% solution. E-BPO/Citric acid phase was added to the emulsion under moderate mixing and circulation then mixing was continued for 15 minutes. The emulsion pH was adjusted to 4 by HCl 10% solution titration. 694.4 grams of encapsulated ATRA 3.6% water suspension were added to 55° C. emulsion during mixing then water was added until the total weight of the emulsion reached 50 kilograms and mixed for 5 minutes. The emulsion continued mixing for 90 minutes during cool down to 30° C.

### Example 2

Table 1 below shows the particle size distribution of eight E-ATRA suspensions (the left side of the Table) and its influence on the stability of the cream formulations (the right side of the table). The eight cream formulations were prepared from the eight E-ATRA suspensions. The differences between the various suspensions is the manufacturer of the critical raw materials. As can be seen from the table the particle size distribution of the suspensions influences the percentage of the ATRA degradant RRT 0.44 (all-trans 5,6-epoxy retinoic acid) and the % degradation of ATRA in the cream formulations after stability of 2 weeks at 40° C./75% RH.

TABLE 1
Influence of particle size distribution (PSD) on stability of the corresponding cream

| | | | E-ATRA, 0.05% & E-BPO, 3% cream | | | | |
|---|---|---|---|---|---|---|---|
| | | | | | 2 w 40° C. | | |
| E-ATRA 3.6% suspension | | | | TZ | | all-trans | |
| | PSD, μm | | | ATRA | ATRA | 5,6-epoxy | % |
| Batch # | d(0.1) | d(0.9) | Batch # | assay | assay | retinoic acid | degradation |
| 450-140-01 | 5 | 49 | 458-014-01 | 0.0506 | 0.0494 | 0.74 | 2.37 |
| 458-007-01 | 12 | 42 | 458-034-01 | 0.0508 | 0.0501 | 0.57 | 1.38 |
| 458-010-01 | 4 | 54 | 458-031-01 | 0.0507 | 0.0492 | 1.14 | 2.96 |
| 458-037-01 | 6 | 41 | 458-043-01 | 0.0515 | 0.0513 | 0.54 | 0.39 |
| 467-089-01 | 13 | 40 | 467-101-01 | 0.0513 | 0.0510 | 0.31 | 0.58 |
| 467-111-01 | 5 | 34 | 467-118-01 | 0.0512 | 0.0514 | 0.24 | 0.00 |
| 467-007-01 | 15 | 41 | 401-146-01 | 0.0508 | 0.0500 | 0.42 | 1.57 |
| 401-138-01 | 5 | 44 | 467-023-01 | 0.0559 | 0.0548 | 0.43 | 1.97 |

PSD—particle size distribution,
TZ—time-zero,
2 w 40° C.—2 weeks at 40° C.

TABLE 2
Stability data of E-ATRA, 0.05% & E-BPO,
3% cream after 3, 18 or 24 months storage

ATRA Assay

| | | 3 m at | 18 m at | 24 m at |
|---|---|---|---|---|
| Batch# | Time zero | 25° C. | 2° C.-8° C. | 2° C.-8° C. |
| P3156- | 0.0494 | 0.0491 | 0.0489 | 0.0486 |
| 00315 | (98.8%) | (98.2%) | (97.8%) | (97.2%) |
| P3156- | 0.0504 | 0.0479 | 0.0479 | 0.0478 |
| 00116 | (100.8%) | (95.8%) | (95.8%) | (95.6%) |

TABLE 3
Stability data of E-ATRA, 0.1% & E-BPO,
3% cream after 3, 18 or 24 months storage

ATRA Assay

| | | 3 m at | 18 m at | 24 m at |
|---|---|---|---|---|
| Batch# | Time zero | 25° C. | 2° C.-8° C. | 2° C.-8° C. |
| P3149- | 0.0973 | 0.0962 | 0.0955 | 0.0943 |
| 00115 | (97.3%) | (96.2%) | (95.5%) | (94.3%) |
| P3149- | 0.099 | 0.0949 | 0.0971 | 0.0954 |
| 00116 | (97.7%) | (94.9%) | (97.1%) | (95.4%) |

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A composition comprising:
microcapsules comprising tretinoin, wherein:
said microcapsules comprise a core containing tretinoin dispersed in an oil and a phase changing material and a metal oxide shell; and
wherein said tretinoin is in solid form and said microcapsules have a D₉₀ between 30 μm to 50 μm; and
wherein said tretinoin is in the range of 0.01%-0.1% by weight of the composition; and
microcapsules comprising solid benzoyl peroxide; wherein said benzoyl peroxide is in an amount of 3% by weight of the composition.

2. The composition according to claim 1, wherein degradation of said tretinoin is less than 2.5% after two weeks storage at 40° C. and 75% relative humidity.

3. The composition according to claim 1, wherein the degradation of said tretinoin is less than 2%.

4. The composition according to claim 1, wherein said tretinoin is in the amount of between 0.05% and 0.1% weight of the composition.

5. The composition according to claim 7, wherein said tretinoin is in the amount of 0.075% weight of the composition.

6. The composition according to claim 1, wherein the composition comprises a carrier, wherein the carrier is in the form of an ointment, a cream, a lotion, an oil, a solution, an emulsion, a gel, a paste, a milk, an aerosol, a powder, or a foam.

7. The composition according to claim 1, wherein the concentration of all-trans 5,6-epoxy retinoic acid after two weeks storage at 40° C. and 75% relative humidity is less than 1% by weight of the initial tretinoin amount prior to storage.

8. The composition according to claim 1, wherein the degradation of said tretinoin is not more than 5% by weight of the initial tretinoin amount, after 18 or 24 months storage at 2° C.-8° C.

9. The composition according to claim 1, wherein the degradation of said tretinoin is not more than 5% by weight of the initial tretinoin amount, after 3 months storage at 25° C. and 60% RH.

10. The composition according to claim 1, wherein the amount of said tretinoin is 0.1% weight of the composition and the amount of said benzoyl peroxide is 3% weight of the composition.

11. The composition of claim 1, wherein said composition has a tretinoin dissolution rate of less than 60% weight/h as measured in a medium of 30%:70% V/V mixture of water and isopropyl alcohol at ambient temperature.

12. The composition according to claim 11, wherein said composition has a tretinoin dissolution rate of less than 40% weight/h.

13. A method for treating a skin disease, disorder or condition selected from acne, infection, inflammation, pruritus, psoriasis, seborrhea, contact dermatitis, rosacea, melasma, photoaging, photodamage, fine wrinkles, and a combination thereof in a subject in need thereof, said method comprising topically administering to said subject a composition according to claim 1.
